(19) United States
(12) Patent Application Publication
(10) Pub. No.: US 2022/0409029 A1
(43) Pub. Date: Dec. 29, 2022

(54) ENDOSCOPE AND ENDOSCOPE SYSTEM

(71) Applicant: HOYA CORPORATION, Tokyo (JP)

(72) Inventors: Akihiko NISHIDE, Tokyo (JP); Kenta KOSUGI, Tokyo (JP)

(73) Assignee: HOYA CORPORATION, Tokyo (JP)

(21) Appl. No.: 17/780,269

(22) PCT Filed: Feb. 3, 2021
(86) PCT No.: PCT/JP2021/004003

§ 371 Date: May 26, 2022

(30) Foreign Application Priority Data
Mar. 27, 2020 (JP) 2020-058551

(51) Int. Cl. A61B 1/045; G02B 23/24

(52) U.S. Cl. CPC A61B 1/045; G02B 23/24

(57) ABSTRACT

An endoscope includes: an image sensor in which a color filters having two or more colors is disposed at an imaging position and has a predetermined array pattern; and a correction processing unit configured to repeatedly perform processing of changing a target pixel value at a target imaging position to a median value between a neighboring pixel value and the target pixel value, while changing the target imaging position, when a pixel value at a neighboring imaging position near the target imaging position including at least two most neighboring imaging positions on a row closest to the target imaging position with the target imaging position interposed therebetween at an imaging position of a color filter, which is on a row along one direction identical to the target imaging position in image data output from the image sensor, and has a same color as a color filter at the target imaging position, is set as a neighboring pixel value.

### TECHNICAL FIELD

The present invention relates to an endoscope that images a biological tissue in a body cavity, and an endoscope system.

### BACKGROUND ART

In the related art, an endoscope is inserted into a body cavity such as a human body, and a lesion of a biological tissue in the body cavity rs examined. In an endoscope system including the endoscope, an endoscope processor performs image processing on a captured image obtained by imaging the biological tissue and then the captured image is displayed as a moving image on a monitor screen (JP 2012-134875 A).

In such an endoscope system, presence or absence of a lesion portion and a degree of progress of the lesion portion are diagnosed by observing the image displayed on the monitor screen, and thus it is preferable to perform processing from the imaging by the endoscope to the image display on the monitor screen in a short time.

For example, since a color filter is provided at an imaging position of an image sensor used in such an endoscope system, the color filter having a predetermined array pattern such as a Bayer array, in order to obtain a three-color image from image data (RAW data) output from the image sensor, it is necessary to calculate pixel values at the imaging positions where colors of the color filter are different by performing interpolation using image values in the image data corresponding to the imaging positions of the same color filter. That is, the pixel values to be calculated are calculated based on surrounding image values. Note that examples of the color filter include, in addition to a color filter of RGB (Red, Green, Blue), a color filter of CYGM (cyan, yellow, green, magenta) and a color filter of RGBE (Red, green, blue, emerald).

### SUMMARY OF INVENTION

### Technical Problem

In such an endoscope, the image sensor may include a defective pixel. The defective pixel refers to a pixel on the captured image corresponding to an imaging position where photoelectric conversion of a light receiving surface of the image sensor does not normally function and an electric signal is not generated in proportion to a light receiving amount. In many cases, the defective pixel has a pixel value specifically lower or higher as compared with the surrounding pixels, and is not formed continuously but formed as an isolated pixel corresponding to one pixel or several pixels. Therefore, the defective pixel is always displayed as, for example, a white point or a black point, and there is a case where an output value of the pixel of the image sensor does not change even when brightness changes.

On the other hand, in the image sensor, in order to generate image data including information of a three-color image (RGB image) for each pixel from primary color image data (RAW data) output from the image sensor including the color filter arrayed in a predetermined array pattern as described above, processing of calculating image values of different color filters corresponding to the imaging positions is performed by performing interpolation using the image values in the image data corresponding to the imaging positions of the same color filter.

However, when this processing is performed on image data in which a defective pixel is present, the pixel value of the defective pixel is diffused to the surrounding pixel values by the interpolation processing, and thus a quality of the three-color image may be deteriorated.

It is also possible to correct the defective pixel by using a median filter having a size of 3 pixels×3 pixels for the three-color image, but it is difficult to completely remove the diffused pixel value of the defective pixel. Furthermore, in a case where the median filter is used, since writing, calling, and the like are performed by using data buffer having three lines, a processing time is taken, and in addition, in consideration of speeding up the processing, it is necessary to prepare a data buffer having a size corresponding to the median filter, which is not preferable also in terms of simplification of hardware.

An object of the present invention is to provide an endoscope capable of efficiently correcting a pixel value of a defective pixel with a small amount of hardware without diffusing the pixel value of the defective pixel in an image sensor around the defective pixel in a color image, and an endoscope system.

### Solution to Problem

An aspect of the present invention is an endoscope that images a biological tissue in a body cavity. The endoscope includes:

an image sensor in which a color filter having two or more different colors is disposed at an imaging position and has a predetermined array patter

a correction processing unit configured to repeatedly perform processing of changing a target pixel value at a target imaging position to a median value between a neighboring pixel value and the target pixel value, while changing the target imaging position, when a pixel value at a neighboring imaging position near the target imaging position including at least two most neighboring imaging positions on a row closest to the target imaging position with the target imaging position interposed therebetween at an imaging position of a color filter, which is on a row along one direction identical to the target imaging position in image data output from the image sensor, and has a same color as a color filter at the target imaging position, is set as a neighboring pixel value; and

a color image data generation unit that generates color image data including image information for each color of the color filter for each pixel after the processing by the correction processing unit.

Another aspect of the present invention is an endoscope that images a biological tissue in a body cavity. The endoscope includes:

an image sensor in which a color filter having two or more different colors is disposed at an imaging position and has a predetermined array pattern;

a correction processing unit configured to repeatedly perform processing of changing a first target pixel value at a first target imaging position in which a variation between a neighboring pixel value and a target pixel value at a target imaging position satisfies at least a predetermined condition to a median value between the neighboring pixel value and the first target pixel value, and maintaining a second target pixel value at a second target imaging position, which does not satisfy the predetermined condition, without changing the second target pixel value while changing the target imaging position, when a pixel value at a neighboring imaging position near the target imaging position including at least two most neighboring imaging positions on a row closest to the target imaging position with the target imaging position interposed there between at an imaging position of a color filter, which is on a row along one direction identical to the target imaging position in image data output from the image sensor, and has a same color as a color filter at the target imaging position, is set as a neighboring pixel value; and

a color image data generation unit that generates color image data including image information for each color of the color filter for each pixel after the processing by the correction processing unit.

It is preferable that the variation is a variance or standard deviation between the neighboring pixel value and the target pixel value, and in the predetermined condition, the variance or the standard deviation is greater than a preset threshold value.

It is preferable that the image sensor is configured to image the biological tissue a plurality of times at regular time intervals, and

the correction processing unit is configured to change the first target pixel value to the median value in a case where the first target imaging position is at the same position between image data obtained by the image sensor performing imaging at least two times at different imaging times.

It is preferable that the imaging of the two times by the image sensor is imaging under different illumination conditions.

It is preferable that the one direction is the same as one array direction of imaging positions at which the image sensor sequentially outputs the image data.

It is preferable that the image sensor performs imaging a plurality of times, and the image sensor switches a scanning direction of the imaging positions at which the image data is sequentially output between two directions orthogonal to each other every time the imaging is performed, and

the one direction is switched between the two directions every time the imaging is performed.

It is preferable that the array pattern is a pattern in which two color filters are alternately disposed on the row.

It is preferable that the neighboring imaging position includes an imaging position that is closest to each of the most neighboring imaging positions and is different from the target imaging position.

Still another aspect of the present invention is an endoscope system including:

the endoscope;

an endoscope processor that performs image processing on an image obtained by imaging of the image sensor; and

a monitor on which the image subjected to the image processing is displayed.

### Advantageous Effects of Invention

According to the endoscope and the endoscope system, a pixel value of a defective pixel can be efficiently corrected with a small amount of hardware without diffusing the pixel value of the defective pixel in an image sensor around the defective pixel in a color image.

### BRIEF DESCRIPTION OF DRAWINGS

FIG. 1 is a block diagram illustrating a configuration of an endoscope system according to an embodiment.

FIG. 2 is a block diagram illustrating a main part of a configuration of a driver signal processing circuit in an endoscope of an embodiment.

FIG. 3 is a diagram illustrating an example of image data output from an image sensor having a color filter in a Bayer array used in an endoscope according to an embodiment.

FIG. 4A is a view illustrating an example of an image displayed in an endoscope system in the relate art.

FIG. 4B is a view illustrating an example of an image displayed in an endoscope system according to an embodiment.

### DESCRIPTION OF EMBODIMENTS

Hereinafter, an endoscope and an endoscope system according to an embodiment will be described in detail.

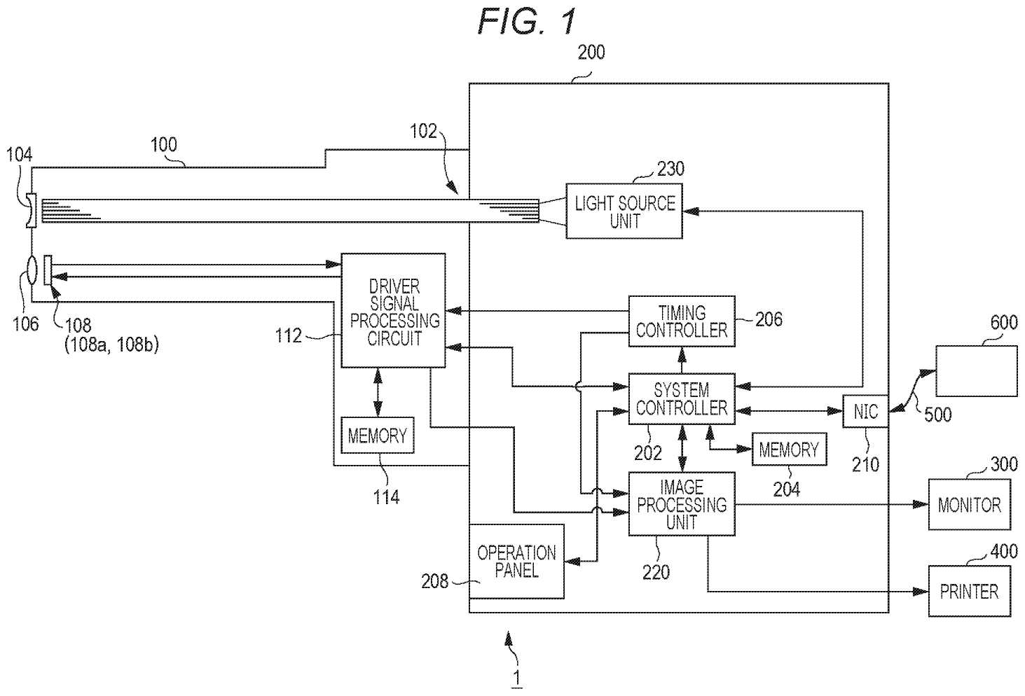

FIG. 1 is a block diagram illustrating a configuration of an endoscope system 1 according to an embodiment.

As illustrated in FIG. 1, the endoscope system 1 includes an endoscope 100, an endoscope processor 200, a monitor 300, and a printer 400.

The endoscope processor 200 includes a system controller 202 and a timing controller 206. The system controller 202 executes various programs stored in a memory 204 and integrally controls the entire endoscope system 1. Furthermore, the system controller 202 changes various settings of the endoscope system 1 in accordance with an instruction of a user (operator or assistant) which is input to an operation panel 208. The timing controller 206 outputs a clock pulse for adjusting an operation timing of each unit to each circuit in the endoscope system 1.

The endoscope processor 200 includes a light source unit 230 that supplies illumination light to the endoscope 100. Although not illustrated, for example, the light source unit 230 includes a high-luminance lamp that emits white illumination light by receiving drive power from a lamp power supply, for example, a xenon lamp, a metal halide lamp, a mercury lamp, or a halogen lamp. The light source unit 230 is configured so that the illumination light emitted from the high-luminance lamp is condensed by a condensing lens (not illustrated) and then incident on an incident end of a light carrying bundle (LCB) 102, which is a bundle of optical fibers of the endoscope 100, via a dimmer (not illustrated).

Alternatively, the light source unit 230 includes a plurality of light emitting diodes that emit light in a wavelength band of a predetermined color. The light source unit 230 is configured so that light emitted from each of the light emitting diodes is synthesized using an optical element such as a dichroic mirror or the like, the synthesized light is condensed as illumination light by the condensing lens (not illustrated), and then incident on the incident end of the light carrying bundle (LCB) 102 of the endoscope 100. A laser diode can be used instead of the light emitting diode. The light emitting diode and the laser diode have features such as low power consumption, a low heat generation amount, and the like, as compared with other light sources, and thus, have an advantage that a bright image can be acquired while suppressing power consumption or a heat generation amount.

Note that in the example illustrated in FIG. 1, the ht source unit 230 is provided by being incorporated in the endoscope processor 200, but may be provided in the endoscope system 1 as a device separate from the endoscope processor 200, Furthermore, the light source unit 230 may be provided at a distal tip of the endoscope 100 to be described later. In this case, the LCB 102 that guides the illumination light is unnecessary.

The illumination light incident from the incident end into the LCB 102 is propagated in the LCB 102, is emitted from an emission end of the LCB 102 disposed in the distal tip of the endoscope 100, and is applied to an object via a light distribution lens 104. Reflected light from the object forms an optical image on a light receiving surface of an image sensor 108 via an objective lens 106.

The image sensor 108 is, for example, a single-plate color charge-coupled device (CCD) image sensor in which various filters such as an infrared (IR) cut filter 108a and a color filter 108b having a Bayer array are disposed on the light receiving surface, the image sensor 108 generating each image data of red (R), green (G), and blue (B) according to the optical image formed on the light receiving surface. Instead of the single-plate color CCD image sensor, a single-plate color complementary metal oxide semiconductor (CMOS) image sensor can be used. In the image sensor 108, the color filter 108b of two or more different colors having a predetermined array pattern is disposed at the imaging position.

A driver signal processing circuit 112 is provided in a connector unit of the endoscope 100 connected to the endoscope processor 200. The driver signal processing circuit 112 generates image signals (luminance signal Y, and color difference signals Cb and Cr) by performing defect correction processing and predetermined signal processing such as matrix calculation on the image data output from the image sensor 108, and outputs the generated image signals to an image processing unit 220 of the endoscope processor 200. Furthermore, the driver signal processing circuit 112 accesses a memory 114 to read specific information of the endoscope 100. The specific information of the endoscope 100 recorded in the memory 114 includes, for example, the number of pixels or sensitivity of the image sensor 108, an operable frame rate, and a model number. The driver signal processing circuit 112 outputs the specific information read from the memory 114 to the system controller 202. As described above, the endoscope 100 captures an image of a biological tissue in a body cavity by using the image sensor 108.

The system controller 202 performs various calculations based on the specific information of the endoscope 100 and generates a control signal. The system controller 202 controls operations and timings of various circuits in the endoscope processor 200 by using the generated control signal so as to perform processing suitable for the endoscope 100 connected to the endoscope processor 200.

The timing controller 206 supplies the clock pulse to the driver signal processing circuit 112, the image processing unit 220, and the light source unit 230 in accordance with timing control of the system controller 202. The driver signal processing circuit 112 performs driving control on the image sensor 108 at a timing synchronized with a frame rate of a video image processed on the endoscope processor 200 side in accordance with the clock pulse supplied from the timing controller 206.

Under control of the system controller 202, the image processing unit 220 generates a video signal for displaying an endoscopic image on a monitor, based on the image signal input from the driver signal processing circuit 112, and outputs the video signal to the monitor 300. The image processing unit 220 obtains an inflammation evaluation value indicated by digitalizing a degree of a degree of inflammation of the biological tissue based on information regarding color components of the image in the image of the biological tissue obtained by the endoscope 100 as necessary, and further generates a color map image in which a pixel evaluation value of each pixel obtained by the digitization processing is replaced with a color. A video signal for displaying information regarding the inflammation evaluation value and the color map image on the monitor 300 is generated and output to the monitor 300. According to this, the operator can accurately perform, for example, evaluation for a degree of inflammation of the target biological tissue through the image displayed on a display screen of the monitor 300. The image processing unit 220 outputs the inflammation evaluation value and the color map image to the printer 400, as necessary.

The endoscope processor 200 is connected to a server 600 via a network interface card (NIC) 210 and a network 500. The endoscope processor 200 can download information regarding an endoscopic examination (for example, electronic medical chart information of a patient or information of the operator) from the server 600. For example, the downloaded information is displayed on the display screen of the monitor 300 or the operation panel 208. Furthermore, the endoscope processor 200 uploads an endoscopic examination result (endoscopic image data, an examination condition, an image analysis result, or an operator's opinion) to the server 600 and thus the endoscopic examination result can be stored in the server 600.

In such an endoscope system 1, as described above, in a case where a defective pixel is present in the image data output from the image sensor 108 that has received light passing through the color filter 108b, when processing for obtaining a color image (RGB image) is performed, the pixel value of the defective pixel is also diffused to the surrounding pixel values by this processing, and thus a quality of a three-color image may deteriorate.

Therefore, the endoscope 100 performs correction processing on primary color image data obtained by receiving light through the color filter 108b before generating color image data (for example, YCbCr signal) including image information for each color of the color filter 108b for each pixel (before demosaic processing). Specifically, the correction processing is performed in the driver signal processing circuit 112 provided in a connector via which the endoscope 100 is connected to the endoscope processor 200.

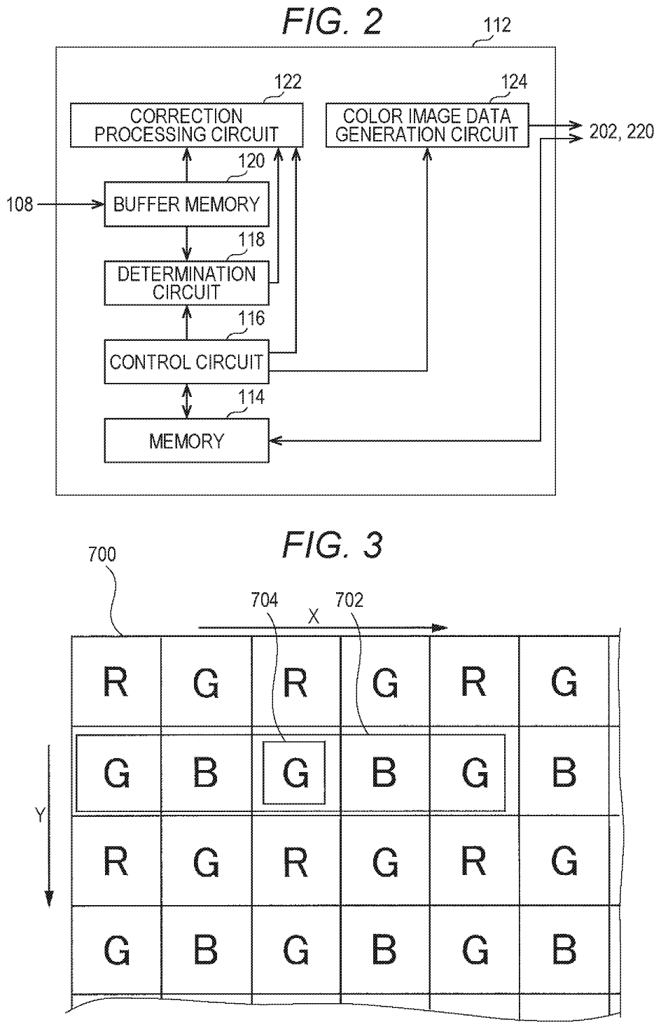

FIG. 2 is a block diagram illustrating a main part of a configuration of the driver signal processing circuit in the endoscope of the embodiment.

The driver signal processing circuit 112 includes a control circuit 116, a determination circuit 118, a buffer memory 120, a correction processing circuit (correction processing unit) 122, and a color image data generation circuit (color image data generation unit) 124, in addition to the memory 114 described above.

The control circuit 116 is communicably connected to the system controller 202, calls information stored in the memory 114 according to an instruction of the system controller 202, transmits the information to the system controller 202, and stores the information transmitted from the system controller 202 as necessary. Moreover, the control circuit 116 is configured to control and manage operations of the determination circuit 118, the correction processing circuit 122,and the color image data generation circuit 124.

The correction processing circuit 122 is a portion configured to perform median filter processing having a set size on a pixel value at a target imaging position while changing the target imaging position in the image data output from the image sensor 108. Specifically, the correction processing circuit 122 sets, as a neighboring pixel value, a pixel value at a neighboring imaging position of a color filter having the same color as that of the color filter at the target imaging position which is identical to the target imaging position and on a row along one direction, and the target pixel value at the target imaging position is changed to a median value between the neighboring pixel value and the target pixel value at the target imaging position, in a pixel array.

The neighboring imaging position includes at least two most neighboring imaging positions on a row closest to the target imaging position with the target imaging position interposed therebetween. In addition to the most neighboring imaging position, the neighboring imaging position preferably also includes an imaging position that is closest to each of the most neighboring imaging positions and is different from the target imaging position.

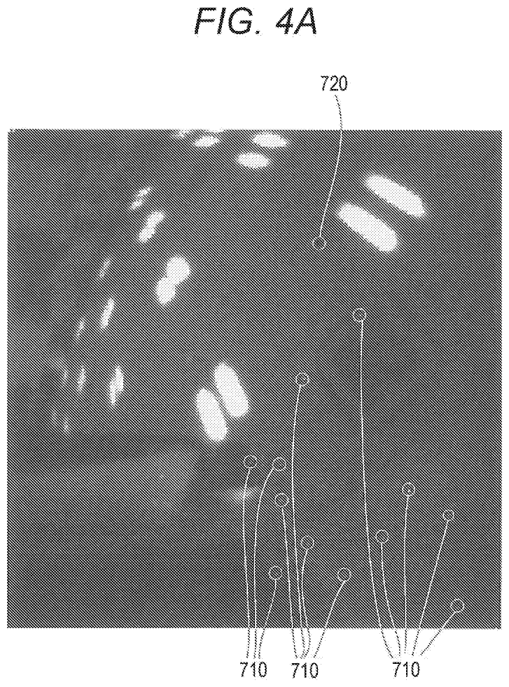

FIG. 3 is a diagram illustrating an example of the image data output from the image sensor having a color filter in a Bayer array used in the endoscope according to the embodiment. According to a pixel array 700 illustrated in FIG. 3, the image sensor 108 alternately outputs R image data and G image data in a row of a first stage, alternately outputs the G image data and B image data in a row shape along a X direction from a left end in a row of a second stage, alternately outputs R image data and the G image data in the row shape along the X direction from the left end in a row of a third stage similarly to the row of the first stage, and alternately outputs the image data and the G image data in the row shape along the X direction from the left end in a row of a fourth stage similarly to the row of the second stage. The R image data is output data that is output by the image sensor 108 when the image sensor 108 receives light transmitting an R filter of the color filter 108b, the G image data is output data that is output by the image sensor 108 when the image sensor 108 receives light transmitting a G filter of the color filter 108b, and the B image data is output data that is output by the image sensor 108 when the image sensor 108 receives light transmitting a B filter of the color filter 108b.

In such image data, in a case where a position of a third G image data from the left end in the second stage is set as the target imaging position, a pixel value at a most neighboring target imaging position close to the third target imaging position among the pixel positions on the row of the second stage having the same G image data, that is, in the example of FIG. 3, a pixel value of each of a first pixel position from the left side in the second stage and a fifth pixel position from the left side in the second stage is set as a most neighboring target pixel value, and a median value between two most neighboring target pixel values and the target pixel value is changed to a target pixel value. Therefore, in such processing, the median value can be set to the target pixel value by performing filter processing by aligning the target imaging position with a central pixel position of a median filter 702. For example, in a case where the median filter 702 has a filter size of five pixels in a lateral direction and one pixel in a vertical direction, the target imaging position is aligned with the pixel position of the third pixel, which is the central pixel position, and the median value among the pixel value of the first pixel, the pixel value of the fifth pixel, and the pixel value of the third pixel which is the central pixel, in the lateral direction, is output and set as the target pixel value.

In this manner, by moving the target imaging position one by one, in the example illustrated in FIG. 3, the median filter 702 is shifted by one imaging position to the right side in the X direction, and the median filter 702 is applied to the target imaging position of the B image data. That is, the direction of the median filter 702 is set n the X direction, and the movement of the median filter 702 is also set in the X direction.

The correction processing circuit 122 obtains a median value for each target imaging position and gives the median value as a target pixel value to the target imaging position.

In the example illustrated in FIG. 3, since the image data output from the image sensor 108 is transmitted in order from the left side of the first stage, the buffer memory 120 is a line buffer memory configured to sequentially store pixels of the number (in the example illustrated in FIG. 3, the target pixel value and four pixel values before and after the target pixel value) corresponding to the filter size of the median filter 702. The buffer memory 120 stores image data by a first-in first-out (FIFO) method every time one piece of image data is transmitted from the image sensor 108. In response to an instruction of the control circuit 116, the stored buffer memory 120 outputs the image data to the correction processing circuit 122. According to this, the median value can be obtained using the pixel value corresponding to the size of the median filter 702 as illustrated in FIG. 3. Therefore, one direction when the target imaging position and a neighboring target imaging position are on the same row along the one direction is preferably the same as one array direction (scanning direction) of the imaging positions at which the image sensor 108 sequentially outputs the image data, from the viewpoint that the image data sequentially sent from the image sensor 108 can be temporarily stored in the buffer memory 120.

In the example illustrated in FIG. 3, the determination circuit 118 is a portion configured to determine whether or not a variation between the target pixel value of the target imaging position, which is the G image data, and a neighboring target pixel value including at least the most neighboring target imaging position separated from the target imaging position by two imaging positions forward and backward of the X direction satisfies a predetermined condition. In a case where a determination result is positive, the control circuit 116 instructs the correction processing circuit 122 to perform the correction processing, and in a case where the determination result is negative, the control circuit 116 instructs the correction processing circuit 122 not to perform the correction processing. Variation between the target pixel value and the neighboring target pixel value is, for example, a difference, a standard deviation, or variance between a maximum value and a minimum value among those pixel values.

Since it is determined whether or not the target imaging position corresponds to the position of the defective pixel in the image sensor, only the pixel value of the defective pixel can be changed to the median value.

The determination circuit 118 stores, in the memory 114, position information of the target imaging position at which the determination result is positive (first target imaging position to be described below).

The color image data generation circuit 124 is a portion configured to generate color image data (luminance signal Y, and color difference signals Cb and Cr) including image information for each color (R, G, or B) of the color filter 108b for each pixel from data corrected by the correction processing circuit 122, that is, image data in which the pixel value of the defective pixel is corrected. The color image data generation circuit 124 performs matrix calculation and the like as necessary when generating the color image data.

In this manner, the endoscope 100 repeatedly performs the correction processing of changing a first target pixel value at the first target imaging position in which the variation between the pixel value at the neighboring imaging position and the target pixel value satisfies at least the predetermined condition to the median value between the neighboring pixel value and the first target pixel value, and maintaining a second target pixel value at a second target imaging position (other than the first target imaging position) that does not satisfy the predetermined condition without changing the second target imaging position while changing the target imaging position. Thereafter, the endoscope 100 generates color image data including image information for each color of the color filter 108b for each pixel from the corrected image data.

Therefore, the pixel value of the defective pixel in the image sensor 108 does not diffuse around the defective pixel in the color image. Moreover, since the neighboring target imaging position is on a row along one direction identical to the target imaging position, the buffer memory 120 only needs to be provided with a memory having a size corresponding to the size (in the example illustrated in FIG. 3, the size of 1×5 pixel) of the median filter 702. Therefore the pixel value of the defective pixel can be efficiently corrected with a small amount of hardware.

In the endoscope system of the related art, when color image data is generated, a defective pixel is corrected using, for example, a median filter having a size of 3×3 pixel. Therefore, there has been a case where a peculiar pixel value in the defective pixel spreads to surrounding pixel values and the size of the defective pixel increases in the color image data. Moreover, since the median filter having a size of 3×3 pixel is used, the line buffer memory cannot be used. It is necessary to perform processing of extracting pixel values for 3×3 pixel, for example while temporarily storing the entire captured image data in a frame memory, and it is necessary to greatly increase hardware devices. In this respect, the endoscope 100 does not need to greatly increase the hardware devices.

According to the device configuration illustrated in FIG. 2, the determination circuit 118 determines whether or not to perform the correction processing in order to find a defective pixel. However, in order to simplify the device configuration and quickly perform the processing, the correction processing circuit 122 may unconditionally perform the correction processing on the image data sequentially transmitted from the image sensor 108 without finding the defective pixel.

Therefore, in this case, the correction processing circuit 122 is configured to repeatedly perform the correction processing of changing the target pixel value at the target imaging position in the image data output from the image sensor 108 to the median value between the neighboring pixel value at the neighboring target imaging position on the same row as the target imaging position and the target pixel value while changing the target imaging position. Thereafter, the endoscope 100 generates color image data including image information for each color of the color filter 108b for each pixel from the corrected image data.

Therefore, also in this case, the pixel value of the defective pixel in the image sensor 108 does not diffuse around the defective pixel in the color image. Moreover, since the neighboring target imaging position is on a row along one direction identical to the target imaging position, the buffer memory 120 only needs to be provided with a memory having a size corresponding to the size (in the example illustrated in FIG. 3, the size of 1×5 pixel) of the median filter 702. Therefore the pixel value of the defective pixel can be efficiently corrected with a small amount of hardware.

The variation between the target pixel value and the neighboring target pixel value, which is used by the determination circuit 118 described above, is preferably a variance or standard deviation between the neighboring pixel value and the target pixel value, and it is preferable that in the predetermined condition described above, the variance or standard deviation is greater than a preset threshold value. By using the variance or the standard deviation, it is possible to quantitatively and reliably indicate that the pixel value as the defective pixel greatly varies, and to more reliably obtain the position of the defective pixel.

Since the image sensor 108 images the biological tissue by scanning the biological tissue a plurality of times at regular time intervals, the image as image data of a moving image is input to the driver signal processing circuit 112 from the image sensor 108, Therefore, it is also preferable that the correction processing circuit 122 (correction processing unit) is configured to change the first target pixel value to the median value in a case where the first target imaging position is at the same position between different image data obtained by the image sensor 108 performing different imaging at least two times. Since the position of the defective pixel is the same position even when the imaging is different, the position of the defective pixel can be reliably obtained by distinguishing the position of the defective pixel from mixing of other noise components in the above-described configuration.

According to the embodiment, it is preferable that the imaging of at least two times at different imaging times by the image sensor 108 is imaging under different illumination conditions. Under different illumination conditions, in a case of the image data having a high pixel value as a whole and in a case of the image data having a low pixel value as a whole, the noise components are generated differently and there is a case where the noise component is not generated in any one of the case of the image data having a high pixel value as a whole and the case of the image data having a low pixel value as a whole. On the other hand, the position of the defective pixel does not change in any cases. Therefore, the position of the defective pixel can be reliably obtained while being distinguished from the mixing of other noise components.

According to the embodiment, in a case where the image sensor 108 performs imaging a plurality of times and the scanning direction of the imaging position in which the image data is sequentially output is switched between two directions orthogonal to each other every time the image sensor 108 performs imaging, it is preferable that one direction when the target imaging position and the neighboring target imaging position are on the same row along one direction is also switched between the two directions every time the image sensor 108 performs imaging in accordance with the switching of the array direction of the imaging positions in which the image data is sequentially output. In the example illustrated in FIG. 3, it is preferable that the direction of the median filter 702 is set to the X direction and the movement of the median filter 702 is also set to the X direction for image data at the time of certain imaging, and the direction of the median filter 702 is set to the Y direction and the movement of the median filter 702 is also set to the Y direction for image data at the time of the next imaging.

In this manner, the scanning direction of the pixel data output from the image sensor 108 is switched between two directions orthogonal to each other every time imaging is performed, and thus the direction of the median filter 702 illustrated in FIG. 3 can also be switched from the X direction to the Y direction in accordance with the switching of the array direction. Therefore, in consecutive images, a decrease in resolution due to the use of the median filter 702 is switched alternately between the X direction and the Y direction. Therefore, it is possible to suppress deterioration in a quality of an image displayed on the monitor 300. In a case where the median filter 702 is used while fixed in one of the X direction and the Y direction, the image quality in one direction is likely to deteriorate.

The color filter 108b in the above-described embodiment may have any array of the color filters, but according to the embodiment, the array pattern of the color filter 108b is preferably a pattern (Bayer array) in which two color filters are alternately disposed on a row as illustrated in FIG. 3. According to this, the most neighboring imaging position is an extremely close position separated from the target imaging position by two imaging positions in the X direction or the Y direction, and thus it is possible to efficiently extract the neighboring target pixel value.

According to the embodiment, the neighboring imaging position preferably includes an imaging position that is closest to each of the most neighboring imaging positions and is different from the target imaging position. That is, it is preferable that the neighboring imaging position includes two most neighboring target imaging positions closest to the target imaging position and two imaging positions closest to each of two most neighboring target imaging positions and not the target imaging positions. Therefore, in this case, it is preferable to obtain the median value by using one target imaging position and at least four neighboring imaging positions. According to this, the position of the defective pixel can be obtained more accurately.

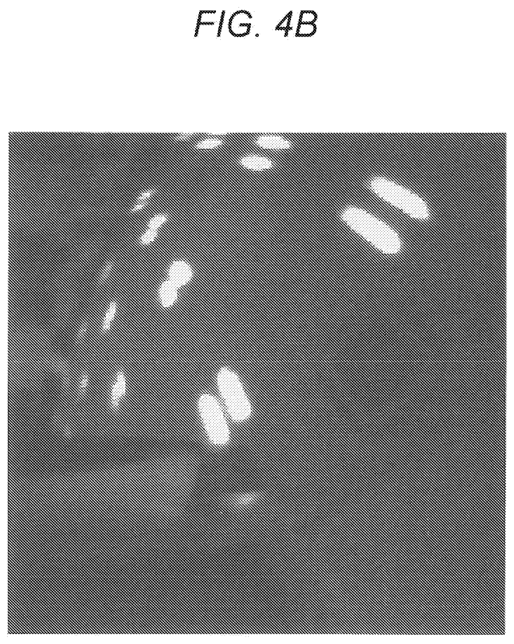

FIG. 4A is a view illustrating an example of an image displayed by the endoscope system of the related art, and FIG. 4B is a view illustrating an example of the image displayed by the endoscope system of the embodiment.

In the display image illustrated in FIG. 4A, six defective pixels 710 displayed in a white dot shape and one defective pixel 720 displayed in a black dot shape are illustrated. In the defective pixel 720, the defective pixel is diffused, and thus a black dot-like area is increased.

On the other hand, in the display image illustrated in FIG. 4B, there are no defective pixels 710 and 720. According to this, an effect of the present embodiment is clear.

Note that a similar effect can be obtained in other color filters such as a CYGM (cyan, yellow, green, and magenta) filter and an RGB (red, green, blue, and emerald) filter.

Hitherto, the endoscope and the endoscope system of the present invention has been described in detail, but the present invention is not limited to the above-described embodiment. As a matter of course, various improvements or modifications may be made within the scope not departing from the concept of the present invention,

1. An endoscope that images a biological tissue in a body cavity, the endoscope comprising:
an image sensor in which a color filter having two or more different colors is disposed at an imaging position and has a predetermined array pattern;
a correction processing unit configured to repeatedly perform processing of changing a target pixel value at a target imaging position to a median value between a neighboring pixel value and the target pixel value, while changing the target imaging position, when a pixel value at a neighboring imaging position near the target imaging position including at least two most neighboring imaging positions on a row closest to the target imaging position with the target imaging position interposed therebetween at an imaging position of a color filter, which is on a row along one direction identical to the target imaging position in image data output from the image sensor, and has a same color as a color filter at the target imaging position, is set as a neighboring pixel value; and
a color image data generation unit that generates color image data including image information for each color of the color filter for each pixel after the processing by the correction processing unit.

2. An endoscope that images a biological tissue in a body cavity, the endoscope comprising:
an image sensor in which a color filter having two or more different colors is disposed at an imaging position and has a predetermined array pattern;
a correction processing unit configured to repeatedly perform processing of changing a first target pixel value at a first target imaging position in which a variation between a neighboring pixel value and a target pixel value at a target imaging position satisfies at least a predetermined condition to a median value between the neighboring pixel value and the first target pixel value, and maintaining a second target pixel value at a second target imaging position, which does not satisfy the predetermined condition, without changing the second target pixel value while changing the target imaging position, when a pixel value at a neighboring imaging position near the target imaging position including at least two most neighboring imaging positions on a row closest to the target imaging position with the target imaging position interposed therebetween at an imaging position of a color filter, which is on a row along one direction identical to the target imaging position in image data output from the image sensor, and has a same color as a color filter at the target imaging position, is set as a neighboring pixel value; and
a color image data generation unit that generates color image data including image information for each color of the color filter for each pixel after the processing by the correction processing unit.

3. The endoscope according to claim 2, wherein
the variation is a variance or standard deviation between the neighboring pixel value and the target pixel value, and
in the predetermined condition, the variance or the standard deviation is greater than a preset threshold value.

4. The endoscope according to claim 2, wherein
the image sensor is configured to image the biological tissue a plurality of times at regular time intervals, and
the correction processing unit is configured to change the first target pixel value to the median value in a case where the first target imaging position is at the same position between different image data obtained by the image sensor performing imaging at least two times at different imaging times.

5. The endoscope according to claim 4, wherein the imaging of the two times by the image sensor is imaging under different illumination conditions.

6. The endoscope according to claim 1, wherein the one direction is the same as one array direction of imaging positions at which the image sensor sequentially outputs the image data.

7. The endoscope according to claim 6, wherein
the image sensor performs imaging a plurality of times, and the image sensor switches a scanning direction of the imaging positions at which the image data is sequentially output between two directions orthogonal to each other every time the imaging is performed, and
the one direction is switched between the two directions every time the imaging is performed.

8. The endoscope according to claim 1, wherein the array pattern is a pattern in which two color filters are alternately disposed on the row.

9. The endoscope according to claim 1, wherein the neighboring imaging position includes an imaging position that is closest to each of the most neighboring imaging positions and is different from the target imaging position.

10. An endoscope system comprising:
the endoscope according to claim 1;
an endoscope processor that performs image processing on an image obtained by imaging by the image sensor; and
a monitor on which the image subjected to the image processing is displayed.
